(19) United States
(12) Patent Application Publication
(10) Pub. No.: US 2015/0168375 A1
(43) Pub. Date: Jun. 18, 2015

(54) CANCER STEM CELLS AND METHODS OF USING THE SAME

(71) Applicant: MEDIMMUNE, LLC, Gaithersburg, MD (US)

(72) Inventors: Haifeng Bao, Gaithersburg, MD (US); Xiaoru Chen, Gaithersburg, MD (US); Patricia Burke, Gaithersburg, MD (US); Xiaoqing Shi, Gaithersburg, MD (US); Sanjoo Jalla, Gaithersburg, MD (US)

(73) Assignee: MEDIMMUNE, LLC, Gaithersburg, MD (US)

(21) Appl. No.: 14/405,536

(22) PCT Filed: Mar. 12, 2013
(86) PCT No.: PCT/US13/30443

§ 371 Date: Dec. 4, 2014

Related U.S. Application Data
(60) Provisional application No. 61/655,245, filed on Jun. 4, 2012.

(51) Int. Cl. G01N 33/50; A01K 67/027

(52) U.S. Cl. CPC G01N 33/5011; A01K 67/0271; G01N 2500/04; A01K 2207/12

(57) ABSTRACT

Provided are methods of culturing cancer stem cells in vitro, where the cancer stem cells have been obtained from the peripheral blood of a patient, and methods of using the cultured cancer stem cells in a xenograft model of cancer and for in vivo and in vitro screening of test compounds. Also provided is an enriched population of cancer stem cells obtained from the peripheral blood of a patient.

### BACKGROUND

Metastasis is a process by which primary tumor cells form a new tumor in a distal organ. Metastasis involves primary cell intravasation, survival in circulation, extravasation, and growth in a distant organ (Mego et al., 2010). Metastasis accounts for 90% of cancer deaths (Weigelt et al., 2005).

Disseminated tumor cells, which include circulating tumor cells (CTC), are thought to serve as the seeds of new tumors (Fidler I J, 2003). These seed cells should have both tumorigenic and metastatic ability. However, there is little direct evidence that human CTCs are tumorigenic and have metastatic potential. Furthermore, the cancer stem cell (CSC) hypothesis suggests that CSCs can be the founder cells of metastasis (Lawson et al. 2009). Although a CSC molecular marker has been detected in breast cancer patient blood, whether CSCs are a subpopulation of CTCs and are responsible for metastasis is unknown. (Kasimir-Bauer S, 2012; Aktas B, 2009)

Tumor cells of human breast cancer patients are heterogeneous; not all cells have the same tumorigenic and metastatic potential (Kang et al., 2003; Liu et al., 2007; Landemaine et al., 2008). A subpopulation of breast cancer tumor cells have been identified as CSCs or cancer initiating cells, cells expressing EpCAM⁺CD44⁺CD24^{dim/-} surface markers (Al-Hajj et al., 2003). Although CSC biology is still evolving and remains controversial, evidence from various studies indicate that these EpCAM⁺CD44⁺CD24^{dim/-} cells are a distinct population that are highly tumorigenic and are associated with breast cancer metastasis (Abraham et al., 2005; Sheridan et al., 2006; Balic et al., 2006; Liu et al., 2007; Liu et al., 2010).

Efforts to evaluate the role of CTCs in metastasis have proven difficult because CTCs represent a rare population of cells, with most metastatic breast cancer (MBC) patients having fewer than 100 CTCs per 7.5 ml of blood. In addition, CTCs are generally fragile and many of them undergo apoptosis and have a short half-life (Meng et al., 2004; Mehes et al., 2001).

Prior to the work described in this application, neither prolonged culture of CTCs (or CSCs obtained therefrom), nor serial clonal passage or transplantation of CTCs (or CSCs obtained therefrom) has been technically feasible (Armstrong et al. 2011). The present disclosure provides such methods and uses for CTCs and CSCs obtained from the same.

### SUMMARY

The present disclosure provides methods of culturing cancer stem cells in vitro, the method comprising incubating in vitro peripheral blood mononuclear cells (PBMCs) from a carcinoma patient, particularly a human patient, in a serum-free culture medium suitable for supporting cancer stem cell maintenance, and maintaining the cell culture in the serum-free medium for at least 5 days and in some instances for up to at least 28 days to obtain an enriched population of cancer stem cells. In one embodiment, flow cytometry is not used to obtain the enriched population of cancer stem cells. In another embodiment, the PBMCs may be treated to remove at least some leukocytes prior to incubating in the serum-free medium. In further embodiments, after culturing in vitro for the at least 5-28 days, the population of cancer stem cells in the cell culture relative to the population of cancer stem cells in PBMCs is enriched at least 10,000-fold. Also provided is an enriched population of cancer stems cells obtained according to the in vitro culturing methods.

In addition, the present disclosure provides methods of forming a human tumor in an immunodeficient, non-human mammal using an enriched population of human cancer stem cells obtained from the PBMCs of a carcinoma patient. Also provided are methods of evaluating the metastatic potential of cancer cells from a carcinoma patient using cancer stem cells obtained from the PBMCs of the carcinoma patient, particularly a human patient, which are injected into an immunodeficient non-human mammal and evaluated to determine the amount of metastatic tumor formation in the immunodeficient non-human mammal.

In another aspect, the present disclosure provides methods of determining the effectiveness of a test compound on reducing the activity or number of cancer stem cells obtained from the PMBCs of a carcinoma patient, particularly a human patient. Such methods can be carried out in vivo in an immunodeficient animal host. In another aspect, the disclosure provides in vitro screening methods for determining the effect of a test compound on in vitro cultured cancer stem cells obtained from the PMBCs of a carcinoma patient, particularly a human patient.

In yet another aspect, the present disclosure provides an enriched population of cancer stem cells, where the cancer stem cells are obtained from the PMBCs of a carcinoma patient, particularly a human patient, and are enriched through in vitro cell culture methods.

### BRIEF DESCRIPTION OF THE DRAWINGS

The accompanying drawings, which are incorporated in and constitute a part of this specification, illustrate certain embodiments, and together with the written description, serve to explain certain principles of the antibodies and methods disclosed herein.

FIG. 1 shows phenotypical analysis of CTCs isolated from breast cancer patients. (a): Identification of putative CSC-like CTCs (EpCAM⁺CD44⁺CD24^{dim/-}CD45⁻) in blood samples from breast cancer patients using confocal and fluorescent microscopy. (b): Flow cytometric analysis of the CSC-like CTC subpopulation in breast cancer patient blood samples.

FIG. 2 shows images and phenotypical analysis of human CTCs grown in vitro. (a): Sphere formation of CTCs in in vitro culture. (b): Detection of CTCs in in vitro culture (28 days) by the CellSearch® (Veridex Corporation, Warrenton, N.J.) method. (c): Phenotypical analysis of CTCs in in vitro culture (9 days), with the majority of CTCs in culture (9 days) showing the CSC (EpCAM⁺CD44⁺CD24^{dim/-}CD45⁻) phenotype by the DepArray™ (Silicon Biosystems, Bologna, Italy) instrument. The two small cells on the bottom are leukocytes for comparison.

FIG. 3 shows that human CTCs initiate tumors after implantation into NOD-SCID Gamma mice. (a): Flow cytometric analysis, showing CTCs in the MBC patient blood sample contained a CSC-like subpopulation (EpCAM⁺CD44⁺CD24^{dim/-}CD45⁻). (b): CTCs in culture before implantation showed the CSC (EpCAM⁺CD44⁺CD24^{dim/-}CD45⁻) phenotype. (c): Tumors developed at the injection sites in two of the three mice who received cultured CTCs. (d): H&E sections of CTC-derived xenograft tumors show histological features of a human breast cancer. (e): Expression of human mammaglobin (MGM) in CTC-derived tumor xenografts. (f): Expression of the human MHC-1 marker in CTC-derived xenograft tumors, but not in adjacent mouse tissue. Images for each stain were taken at the same magnification. Scale bar represents 50 μm for H&E and other stains.

FIG. 4 shows the gene expression analysis of MBC patient CTCs in culture before implantation.

FIG. 5 shows the development of spontaneous metastasis in mice after implantation of human CTCs. (a): H&E sections of mouse lungs show invasion of tumor cells (arrows). (b): Expression of the human MHC-1 marker in tumor cells invaded in mouse lungs (IHC). Images for each stain were taken at the same magnification. Scale bar represents 50 μm for both H&E and hMHC-1 stains. (c): Detection of human mammaglobin A (hMGA) mRNA in CTC-derived tumor xenografts and mouse lungs by qRT-PCR. (d), (e): Detection of CTCs in peripheral blood of tumor-bearing mice, but not in the mouse without tumor.

FIG. 6 shows phenotypical analysis of CTC-derived tumor xenografts. (a): Xenograft tumor cells did not express mouse MHC class I marker H-2Kd. (b), (c): Xenograft tumor cells exhibited phenotypic heterogeneity, including a small fraction of cells with the CSC (EpCAM⁺CD44⁺CD24^{dim/-}) phenotype.

FIG. 7 relates to serial transplantation of CTC-derived tumors in Beige Nude XID mice. (a): Representative tumors (3 months) in mice that were implanted with the CTC-derived tumor xenograft tissue. (b): The secondary-passage tumor xenografts resembled the original CTC-derived tumor xenografts. (c): Tumor cell invasion in lungs of mice implanted with the CTC-derived tumor xenograft tissue. Images for each stain were taken at the same magnification. Scale bar represents 50 um both H&E stains. (d): Detection of CTCs in peripheral blood of mice that were implanted with the CTC-derived tumor xenograft tissue.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

### 1. Definitions

In order that the present invention may be more readily understood, certain terms are first defined. Additional definitions and embodiments encompassed by the terms and definitions herein are set forth throughout the detailed description.

The term “cancer stem cell” refers to a tumorigenic cell with the following phenotype: EpCAM⁺CD44⁺CD24^{dim/-}.

The term “isolated” refers to a cell that is removed from its natural environment, such as the peripheral blood.

The term “patient” refers to any mammalian subject diagnosed with or suspected of having cancer. Peripheral blood mononuclear cells may be obtained from a patient. A patient, in particular, may be a human.

The term “tumorigenic” refers to the ability of a cancer cell to form a tumor when injected into an immunodeficient host animal.

It should be noted that, as used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is also understood that wherever embodiments are described herein with the language “comprising,” otherwise analogous embodiments described in terms of “consisting of” and/or “consisting essentially of” are also provided.

### 2. Carcinoma

A carcinoma is a tumor that arises from an epithelial cell, including but not limited to breast, lung, prostate, colon, pancreas, renal, liver, stomach, brain, head and neck, and rectum tumors. In one embodiment of the methods or enriched populations of cancer stem cells described in this application, the carcinoma patient has breast, lung, prostate, colon, pancreas, stomach, renal, liver, or rectum cancer. In another embodiment, the carcinoma patient has breast, lung, renal, liver, or prostate cancer. In another embodiment, the carcinoma patient has breast, lung, or prostate cancer. In another embodiment, the carcinoma patient has breast or lung cancer. In yet another embodiment, the carcinoma patient has breast cancer.

### 3. Circulating Tumor Cells (CTCs)

CTCs are a population of cancer cells that detach from a primary tumor and enter the circulation. CTCs are very rare cells surrounded by billions of hematopoietic cells (e.g., red and white blood cells, granulocytes, macrophages, neutrophils, basophils) in the peripheral blood.

CTCs can be detected using known techniques, including, but not limited to, CellSearch® (Veridex Corporation, Warren, N.J.) system, AdnaTest, epithelial immunospot (EPISPOT) assay, CTC chip/microchip, laser scanning cytometry MAINTRAC® (Vermont Systems, Inc. Essex Junction, Vt.), fiber-optic array scanning technology (FAST), MagSweeper® (Illumina Inc. San Diego, Calif.) (Mego et al. 2010). Of the techniques, the CellSearch® (Veridex Corporation, Warren, N.J.) system is the only FDA-approved CTC detection system.

### 4. Cancer Stem Cells (CSCs)

A subpopulation of breast cancer tumor cells expressing surface markers of EpCAM⁺CD44⁺CD24^{dim/-} has been identified as CSCs or cancer initiating cells (Al-Hajj et al., 2003) in breast cancer tumors. CSCs have also been prospectively identified from other solid tumors, including colorectal, brain, colon, head and neck, and pancreatic cancer (Dalerbra et al. 2007). As demonstrated for this first time in this application, a certain percentage of CTCs have the CSC phenotype (EpCAM⁺CD44⁺CD24^{dim/-}) and are able to seed tumor xenografts when injected into an immunodeficient animal. As noted above, CTCs represent a rare population of cells in the peripheral blood. CTCs that have a CSC phenotype represent an even rarer population of cells in the peripheral blood and, therefore, prior to the work described in this application, it was not possible to demonstrate that this population of CSC-like cells existed in the peripheral blood and had the ability to seed metastatic tumors.

EpCAM is an epithelial cell adhesion molecule expressed on the surface of epithelial cells, including most carcinomas. CD44 is a cell surface glycoprotein involved in cell-cell interactions, cell adhesion and migration. This protein participates in a wide variety of cellular functions including lymphocyte activation, recirculation and homing, hematopoiesis (formation of blood cellular components), and tumor metastasis. CD24 is a cell surface marker expressed on the surface of B lymphocytes and granulocytes.

Thus, CSCs can be characterized by their cell surface markers, such as EpCAM, CD44, and CD24. These cell surface markers can be identified using reagents that specifically bind to the cell surface molecules, such as antibodies that specifically recognize the cell surface markers. CSCs, therefore, can be identified or selected by positive selection of cell surface markers using known techniques, including immunohistochemistry and fluorescent activated cell sorting (FACS). In addition, it is also possible to eliminate cells from a sample that are not cancer stem cells, using cell surface molecules that are not present on cancer stem cells but are present on other cells in the blood. For example, CD45 is a cell surface marker found on white blood cells and can be used as a negative marker to remove leukocytes from a sample of peripheral blood cells.

Identifying and selecting cells based on the expression of cell surface markers is routine in the art. For example, it is possible to identify and select cells based on the expression of cell surface markers using flow cytometry techniques. Practical Flow Cytometry, Howard Shapiro, Fourth Ed., 2003, John Wiley and Sons, N.J. However, as noted previously, prior to the disclosure of this application, it was not possible to culture in vitro CTCs or CSCs that had been isolated from the peripheral blood using flow cytometry.

### 5. Methods of Culturing Cancer Stem Cells In Vitro

It is possible to grow various different types of cells or cell lines in vitro by mixing the cells or cell lines with a cell culture medium under controlled conditions. In general, cells are grown and maintained in an appropriate environment, such as a cell incubator. The cell incubator maintains appropriate conditions for cell growth, including temperature, humidity, and carbon dioxide and oxygen content (gas mixture). Cell culture conditions vary for different types of cells. Some cells, such as CTCs, are not easily cultured in vitro.

A cell culture medium is composed of a number of ingredients and these ingredients vary from one culture medium to another. The ingredients provide an osmotic force to balance the osmotic pressure across the cell membrane (or wall). Additionally the ingredients provide nutrients for the cell. Some nutrients will be chemical fuel for cellular operations; some nutrients may be raw materials for the cell to use in anabolism; some nutrients may be machinery, such as enzymes or carriers that facilitate cellular metabolism; some nutrients may be binding agents that bind and buffer ingredients for cell use or that bind or sequester deleterious cell products, some nutrients may be growth factors, cytokines, and hormones that regulate normal cellular activities such as cell viability, proliferation, and differentiation.

Depending on the cell and the intended use of the cell, the ingredients of the cell culture medium will optimally be present at concentrations balanced to optimize cell culture performance. Performance will be measured in accordance with a one or more desired characteristics, for example, cell number, cell mass, cell density, O₂ consumption, consumption of a culture ingredient, such as growth factors, cytokines, hormones, glucose or a nucleotide, production of a biomolecule, secretion of a biomolecule, formation of a waste product or by product, e.g., a metabolite, activity on an indicator or signal molecule, etc. Each of the ingredients or a selection thereof will thus preferably be optimized to a working concentration for the intended purpose.

Serum, the supernatant of clotted blood, can be used in cell culture medium to provide components that promote cell growth and/or productivity. These serum components include attachment factors, micronutrients (e.g., trace elements), growth factors (e.g., hormones, proteases), and protective elements (e.g., antitoxins, antioxidants, antiproteases). Serum is available from a variety of animal sources including bovine or equine. When included in cell culture medium, serum is typically added at a concentration of 5-10%.

On the other hand, certain cell culture media are serum free. In these serum-free media, serum can be replaced with defined hormone cocktails, such as HITES or ITES, which contain hydrocortisone, insulin, transferrin, ethanolamine, and selenite. Alternatively, the serum-free media can contain growth factor extracts from endocrine glands, such as epidermal or fibroblast growth factors. Serum-free media can also contain other components as a substitute for serum, including purified proteins (animal or recombinant), peptones, amino acids, inorganic salts, and animal or plant hydrolysates (or fractions thereof).

Any cell culture media that supports the growth of CSCs can be used in the methods described in this application. In one embodiment, the cell culture medium does not contain any serum (i.e., serum-free cell culture medium). Exemplary CSC cell culture media include, without limitation, MammoCult® (Stem Cell Technologies, Vancouver, Canada), mTeSR™1 and mTeSR™2 (Stem Cell Technologies, Vancouver, Canada), Human Breast Cancer Stem Cell Serum Free Media, M36102-29-P (Celprogen, San Pedro, Calif.), EpiCult®C Human Medium (Stem Cell Technologies, Vancouver, Canada), or NeuroCult® NS-A Basal Medium (Stem Cell Technologies, Vancouver, Canada).

MammoCult® (Stem Cell Technologies, Vancouver, Canada) is a serum-free liquid culture medium optimized for the culture of mammospheres from normal human primary breast tissues and tumorspheres from human breast cancer cell lines. It may be supplemented, for example, with MammoCult® (Stem Cell Technologies, Vancouver, Canada) proliferation supplements.

mTeSR™1 (Stem Cell Technologies, Vancouver, Canada) and mTeSR™2 (Stem Cell Technologies, Vancouver, Canada) are complete cell culture media designed for the culture of human induced pluripotent stem cells (hiPSC) and human embryonic stem cells (hESC) in serum-free, feeder-independent conditions. mTeSR®1 (Stem Cell Technologies, Vancouver, Canada) contains BSA and has been shown to maintain hiPSC and hESC pluripotency after extended periods in culture and can also support the derivation of hiPSC. mTeSR™2 (Stem Cell Technologies, Vancouver, Canada) is similar to mTeSR™1 (Stem Cell Technologies, Vancouver, Canada) but has the added advantage of being free of non-human proteins. mTeSR™1 (Stem Cell Technologies, Vancouver, Canada) and mTeSR™2 (Stem Cell Technologies, Vancouver, Canada) may be supplemented, for example, with 5×mTeSR™1 (Stem Cell Technologies, Vancouver, Canada) or 5× mTeSR™2 (Stem Cell Technologies, Vancouver, Canada) supplement (containing recombinant human basic fibroblast growth factor and recombinant human transforming growth factor β).

EpiCult®-C(Stem Cell Technologies, Vancouver, Canada) Human Medium is a serum-free liquid culture medium optimized for the short term culture of human mammary luminal and myoepithelial cells. It may be supplemented, for example, with EpiCult®-C(Stem Cell Technologies, Vancouver, Canada) proliferation supplements.

NeuroCult® (Stem Cell Technologies, Vancouver, Canada) NS-A Basal Medium is a serum-free medium for the culture of human neural stem and progenitor cells. NeuroCult® (Stem Cell Technologies, Vancouver, Canada) NS-A Basal Medium should be supplemented with appropriate cytokines (e.g., epidermal growth factor and basic fibroblast growth factor). The complete medium (containing cytokines) has been optimized to maintain human neural stem cells in culture for extended periods of time without the loss of their self-renewal, proliferation or differentiation potential. NeuroCult® (Stem Cell Technologies, Vancouver, Canada) NS-A Basal Medium may be supplemented using, for example, NeuroCult® (Stem Cell Technologies, Vancouver, Canada) NS-A Proliferation Supplement.

Thus, one aspect of this disclosure is directed to methods of culturing cancer stem cells in vitro. In particular, one embodiment comprises a method of culturing cancer stem cells in vitro, the method comprising (a) preparing a cell culture by incubating in vitro peripheral blood mononuclear cells (PBMCs) in a serum-free cell culture medium suitable for supporting cancer stem cell maintenance, wherein the PBMCs are obtained from a carcinoma patient and comprise cancer stem cells; and (b) maintaining the cell culture in the serum-free cell culture medium for at least 5 days, at least 6 days, at least 7 days, at least 8 days, at least 9 days, at least 10 days, at least 12 days, at least 14 days, at least 16 days, at least 18 days, at least 20 days, at least 22 days, at least 24 days, at least 26 days, at least 28 days, or greater than 28 days, to obtain an enriched population of cancer stem cells. In one embodiment, flow cytometry is not used to obtain the enriched population of cancer stem cells. In a further embodiment, the PBMCs may be treated to remove leukocytes or a portion thereof prior to incubation in the serum-free cell culture media.

In another embodiment, after culturing in vitro for at least 9 days, the cancer stem cells are enriched at least 5000 fold, at least 7500 fold, at least 8000 fold, at least 9000 fold, at least 10000 fold, at least 12500 fold, at least 15000 fold, at least 20000 fold, or at least 25000 fold, or at least 50,000 fold relative to the concentration of the cancer stem cells in PBMCs of the carcinoma patient. In another embodiment, after culturing in vitro for at least 9 days, at least 1%, at least 2%, at least 3%, at least 4%, at least 4.5% or at least 5% of the live cells in the enriched culture are cancer stem cells, as measured by CellSearch. Another aspect is directed to an enriched population of cancer stem cells obtained according to the in vitro culture methods described in this application.

### 5. Enriched Population of Cancer Stem Cells

An enriched population of CSCs is one that has a higher concentration of cancer stem cells, as compared to concentration of the CSCs in the peripheral blood from which they were obtained. Notably, however, enriching CSCs from the peripheral blood using flow cytometry in combination with immunomagnetic separation techniques is problematic, because it was discovered, as discussed in the examples, that CSCs did not grow in cell culture following separation by flow cytometry. Rather, the enrichment of CSCs occurs after peripheral blood cells are cultured in vitro in serum-free cell culture medium suitable for supporting cancer stem cell maintenance. For example, if a peripheral blood sample contains CSCs at a ratio of 1:5×10⁸ cells while CSCs obtained from peripheral blood and cultured in vitro for at least 5-9 days are present at a concentration of 1:100 the CSCs are enriched 5×10⁶ fold. If a leukocyte or PBMC sample contains CSCs at a ratio of 10:1×10⁷ cells per mL while CSCs obtained from leukocytes or PBMCs that have been cultured in vitro for at least 5-9 days are present at a concentration of 1:100, the CSCs are enriched around 10,000 fold. If a leukocyte or PBMC sample contains CSCs at a ratio of 10:1×10⁷ cells per mL while CSCs obtained from leukocytes or PBMCs that have been cultured in vitro for at least 5-9 days are present at a concentration of 1:20, the CSCs are enriched around 50,000 fold.

In one aspect, the disclosure provides an enriched population of cancer stem cells, where the cancer stem cells are obtained from the peripheral blood of a carcinoma patient and where they are enriched as compared to the concentration of the cancer stem cells in the peripheral blood of the carcinoma patient from which they were obtained. In one embodiment the cancer stem cells are human cancer stem cells. In another embodiment, the cancer stem cells are enriched 10,000 to 250,000 fold as compared to the concentration of cancer stem cells in the peripheral blood from which they were obtained. In another embodiment, the enriched population of cancer stem cells comprises at least 1%, at least 2%, at least 3%, at least 4%, at least 5% of the live cells in an enriched cell culture as measured by CellSearch, following in vitro culturing of the peripheral blood of a carcinoma patient. In yet another embodiment, flow cytometry is not used to enrich the CSCs from the peripheral blood. In a further embodiment, the PBMCs may be treated to remove leukocytes or a portion thereof prior to incubation in the serum-free cell culture media.

### 6. Xenograft Model of Cancer

The ability to obtain a population of cells enriched for CSCs from peripheral blood in vitro allows for cultured CSCs to be used to establish tumors in a host animal. The host animal can be any mammal. In one embodiment, the host animal is a laboratory mammal, including, but not limited, to a mouse, a rabbit, a rat, or a primate.

In one aspect, using an immunodeficient host, a population of cells enriched for CSCs from a first species can be used to establish tumors in the immunodeficient host animal of a second species that is different from the first species. Transplanting cells from one species into a host animal belonging to a different species is referred to as a xenograft (derived from the Greek word “xenos,” meaning foreign) and such xenograft models remain the gold standard for testing new approaches to treating cancer. Normally, the immune system of the host animal would mount an immune response against foreign cells from a different species. However, an immunodeficient host animal does not have properly functioning immune system. Because the immunodeficient host animal cannot mount an immune response against the transplanted cancer cells, the cancer cells, if they possess tumorigenic activity, can establish a solid tumor of foreign origin in the host animal. Thus, by way of example, an immunodeficient mouse will not reject human tumor cells. Immunodeficient mice, such as nude mice, severe combined immunodeficiency (SCID) mice, X-linked immunodeficiency mice, are readily available and have been used extensively as hosts for xenograft models of cancer. (Brehm M A et al. 2010). To serve as a xenograft model of cancer, a population of cells enriched for CSCs are injected into the immunodeficient host animal and the host animal is observed for tumor formation. The population of cells enriched for CSCs can be injected into the host animal using any method known in the art. The population of cells enriched for CSCs may be obtained from the peripheral blood of a carcinoma patient following in vitro culture. Typically about 100-1000 of cells of the CSC-enriched culture are injected into the host animal. In certain embodiments, about 100-500, 100-250, 250-500, 500-1000, 500-750, or 750-1000 cells of the CSC-enriched culture are injected into the host animal.

Accordingly, one aspect is directed to a method of forming a human tumor in an immunodeficient non-human mammal, the method comprising injecting an enriched population of human cancer stem cells into the immunodeficient non-human mammal, wherein the enriched population of human cancer stem cells were obtained from the peripheral blood of the carcinoma patient and wherein the injected cells form the human tumor in the immunodeficient non-human mammal.

In one embodiment, the method of forming a human tumor in an immunodeficient non-human mammal further comprises before the injection step: (a) preparing a cell culture by incubating peripheral blood mononuclear cells (PBMCs) obtained from the carcinoma patient in a serum-free cell culture medium suitable for supporting cancer stem cells in culture, wherein the PBMCs comprise human cancer stem cells; and (b) maintaining the cell culture in the serum-free cell culture medium for at least 5 days, at least 6 days, at least 7 days, at least 8 days, at least 9 days, at least 10 days, at least 12 days, at least 14 days, at least 16 days, at least 18 days, at least 20 days, at least 22 days, at least 24 days, at least 26 days, at least 28 days, or greater than 28 days, to obtain the enriched population of human cancer stem cells. In one embodiment, flow cytometry is not used to obtain the enriched population of human cancer stem cells. In a further embodiment, the PBMCs may be treated to remove leukocytes or a portion thereof prior to incubation in the serum-free cell culture media.

In yet another embodiment, the method further comprises a step of isolating the human tumor formed in the immunodeficient non-human mammal and injecting cancer cells obtained from the isolated human tumor into a second immunodeficient non-human mammal, wherein the injected cancer cells obtained from the isolated human tumor form a new human tumor in the second immunodeficient non-human mammal.

Another aspect is directed to a method of evaluating the metastatic potential of cancer stem cells from a carcinoma patient, the method comprising:
a. injecting an enriched population of cancer stem cells from the carcinoma patient into an immunodeficient non-human mammal, wherein the cancer stem cells were obtained from the peripheral blood of the carcinoma patient;
b. determining the metastases in the immunodeficient non-human mammal; and
c. evaluating the metastatic potential of cancer stem cells from the carcinoma patient based on the metastases in the immunodeficient non-human mammal

In one embodiment, the method of evaluating the metastatic potential of cancer stem cells from a carcinoma patient further comprises before the injection step: (a) preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells; and (b) maintaining the cell culture in the serum-free cell culture medium for at least 5 days, at least 6 days, at least 7 days, at least 8 days, at least 9 days, at least 10 days, at least 12 days, at least 14 days, at least 16 days, at least 18 days, at least 20 days, at least 22 days, at least 24 days, at least 26 days, at least 28 days, or greater than 28 days, to obtain the enriched population of cancer stem cells.

Yet another aspect is directed to a method for determining the effectiveness of a test compound on reducing the number or activity of cancer stem cells from a carcinoma patient, the method comprising:
a. injecting an enriched population of cancer stem cells from the carcinoma patient into an immunodeficient non-human mammal, wherein the cancer stem cells were obtained from the peripheral blood of the carcinoma patient;
b. administering the test compound to the immunodeficient non-human mammal before, after, or at the same time as the cancer stem cells are injected into the immunodeficient non-human mammal;
c. determining the amount of tumor formation, metastasis, or stem cell number in the immunodeficient non-human mammal; and
d. comparing the amount of tumor formation, metastasis, or stem cell number in the immunodeficient non-human mammal to a control non-human mammal, wherein a reduction in the amount of tumor formation, metastasis, or stem cell number in the immunodeficient non-human mammal as compared to the control non-human mammal indicates that the test compound is effective to reduce the tumorigenicity metastasis, or stem cell number of the cancer stem cells from the carcinoma patient.

In one embodiment, the method for determining the effectiveness of a test compound on reducing the tumorigenicity of cancer stem cells further comprises before the injection step: (a) preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells; and (b) maintaining the cell culture in the serum-free cell culture medium for at least 5 days, at least 6 days, at least 7 days, at least 8 days, at least 9 days, at least 10 days, at least 12 days, at least 14 days, at least 16 days, at least 18 days, at least 20 days, at least 22 days, at least 24 days, at least 26 days, at least 28 days, or greater than 28 days, to obtain the enriched population of cancer stem cells.

In certain embodiments of the methods discussed in this section, after culturing in vitro for at least 9 days, cancer stem cells are enriched 10,000 to 250,000 fold as compared to the concentration of cancer stem cells in the peripheral blood from which they were obtained. In other embodiments, flow cytometry is not used to obtain the enriched population of cancer stem cells. In yet another embodiment, the cancer stem cells are human cancer stem cells.

The test compound can be any agent that may have an effect on a tumor cell, including, but not limited to a chemical compound, a protein, a nucleic acid, a carbohydrate, a virus, lipid, an antibody, or any other substance. The test compound may be a drug authorized for sale to treat cancer by a regulatory authority or may be an investigational compound that may or may not be in clinical trials.

Amount of tumor formation can be determined by any method known in the art, for example, by determining size of tumor. The size of tumor formed in an animal treated with test compound versus size of tumor formed in a control animal, e.g., an animal that is untreated or treated with placebo, can be compared. A tumor of smaller size in the animal treated with the test compound relative to the control animal indicates that the test compound is effective to reduce the tumorigenicity of the cancer stem cells. A test compound that is effective to reduce the tumorigenicity of the cancer stem cells may form tumors that are at least 10%, at least 20%, at least 25%, at least 30%, at least 50%, at least 75%, at least 80%, or at least 90% smaller by weight or by volume in an animal treated with the test compound than a control animal.

Amount of tumor formation can also be determined by ascertaining the minimum number of cells from an enriched population of cancer stems cells, as described herein, that are required to form tumors in a defined percentage (e.g., 10%, 20%, 30%, 40%, 50%, 60%, 70%, 80%, 90%, or 100%) of animals that have been administered a test compound relative to control animals, e.g., animals that have not been administered the test compound. For example, if 500 cells of an enriched population of cancer stems cells formed tumors in 50% of control animals, while 5000 cells were required to form tumors in 50% of test animals that have been administered the test compound, then the test compound is effective at reducing tumorigenicity of the cells.

Metastasis or metastatic potential can be determined by any method known in the art. Tumor metastases can be identified, for example, by imaging techniques. Imaging techniques that can be used for identifying metastases include ultrasound, CT scans, bone scans, magnetic resonance imaging, and positron emission tomography. A test compound that is effective at reducing metastasis may reduce the total number of metastatic lesions in an animal to which the test compound is administered relative to a control animal. A test compound may, jointly or separately, be effective at reducing metastases if it decreases the number of different organs in which metastatic lesions in an animal are identified.

Number of cancer stem cells can also be determined by any method known in the art. The number of cancer stem cells can be determined, for example, by any of the methods described herein at paragraphs 25, 30-33, and in the Materials and Methods, and Examples. A test compound may be effective if it reduces the number of cancer stem cells in an animal treated with a test compound relative to a control animal if it reduces the number of cancer stems in either the tumor or the circulation by at least 5%, at least 10%, at least 15%, at least 20%, at least 25%, at least 30%, at least 40%, at least 45%, at least 50%, at least 60%, at least 75%, at least 80%, at least 90%, or at least 95%.

The test compound may be administered to the animal before, at the same time or after the enriched population of cancer stem cells is injected in an animal. If the test compound is administered to the animal before the enriched population of cancer stem cells, it may be administered at any time including at least one month, at least three weeks, at least 2 weeks, at least 1 week, at least 5 days, at least 3 days, or at least 1 day before injection of the enriched population of cancer stem cells. If the test compound is administered to the animal at the same time as the enriched population of cancer stem cells, it may be administered to the animal on the same day in the injection with the enriched population of cancer stem cells, or on the same day but separate from the injection with the enriched population of cancer stem cells. If the test compound is administered to the animal in the same injection as the enriched population of cancer stem cells it may be because the enriched population of cancer stem cells has been preincubated for a period of at least 1 day, at least 2 days, at least 3 days, at least 5 days, at least 1 week, at least 10 days, or at least 2 weeks with the test compound. If the test compound is administered to the animal after injection with the enriched population of cancer stem cells, the test compound may be administered before or after tumor formation in the animal by the enriched population of cancer stem cells. The test compound may be administered repeatedly to the animal, for example, once a day, once every 2 days, once every 3 days, once weekly, once every other week, once every three weeks, or once a month.

### 7. In Vitro Screening Assays

In addition to the xenograft cancer model, the effect of a test compound on CSCs can also be measured in vitro. Thus, one aspect is directed to an in vitro screening method for measuring the effect of a test compound on a cell population enriched for cancer stem cells from a carcinoma patient, the method comprising: (a) adding the test compound to an in vitro culture of cancer stem cells, wherein the cancer stem cells were obtained from the peripheral blood of the carcinoma patient; and (b) measuring the effect of the test compound on the cancer stem cells. In one embodiment, the in vitro screening method further comprises before adding the test compound to the in vitro culture of cancer stem cells: (a) preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells; and (b) maintaining the cell culture in the serum-free cell culture medium for at least 5 days, at least 6 days, at least 7 days, at least 8 days, at least 9 days, at least 10 days, at least 12 days, at least 14 days, at least 16 days, at least 18 days, at least 20 days, at least 22 days, at least 24 days, at least 26 days, at least 28 days, or greater than 28 days, to obtain an enriched population of cancer stem cells. In another embodiment, after culturing in vitro for at least 9 days, cancer stem cells are enriched 10,000 to 250,000 fold as compared to the concentration of cancer stem cells in the peripheral blood from which they were obtained. In another embodiment, flow cytometry is not used to obtain the enriched population of cancer stem cells. In yet another embodiment, the cancer stem cells are human cancer stem cells. In yet a further embodiment, prior to incubating the PBMCs in the serum-free media, the PBMCs are treated to remove leukocytes.

The test compound can be any agent that may have an effect on a tumor cell, including, but not limited to a chemical compound, a protein, a nucleic acid, a carbohydrate, a virus, lipid, an antibody, or any other substance. The test compound can be added at any time during the in vitro culturing of the cancer stem cells. Thus, in one embodiment, the test compound is added when the PBMC cell culture is initiated. Alternatively, the test compound can be added at any time during the first through ninth day of the cell culture, or at any time after the ninth day of cell culture.

These methods can be used to screen, for example, potential therapeutic compounds with anti-cancer activity. These methods can also be used to identify an appropriate therapeutic agent for a particular individual whose cancer stem cells comprise the enriched population of cancer stem cells.

The effect of the test compound on the enriched population of cancer stem cells may be measured by a change in number of cancer stem cells in the in vitro culture. It can also be measured by examining characteristics of the CSCs using, for example, microscopy. The effect of the test compound on nucleic acid or protein expression in the CSCs can be measured using techniques available in the art to measure nucleic acid or protein expression, e.g., determining Ki-67 expression as a marker of cell proliferation. Alternatively, the CSCs treated with the test compound in vitro can be injected into an immunodeficient host animal, and the ability of the transplanted CSCs to induce tumors in the host animal measured (see Xenograft Model of Cancer above).

### EXAMPLES

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### Materials and Methods

### 1. Primary CTC Culture

Blood samples obtained from breast cancer patients, consented according to the Human Biological Samples Policy, were purchased from Conversant Biologics. PMBCs were prepared from blood samples using human Lympholyte®-H cell separation medium (Cedar Lane Labs, Burlington, N.C.) and the cells from buffy coat were aliquoted to 6-well ultra-low attachment plates (Corning Inc., Corning, N.Y.) with either 1) MammoCult® (Stem Cell Technologies, Vancouver, Canada), supplemented with MammoCult® (Stem Cell Technologies, Vancouver, Canada) proliferation supplements, or 2) mTeSR™1 (Stem Cell Technologies, Vancouver, Canada) (basal medium+5× supplement of recombinant human basic fibroblast growth factor and recombinant human transforming growth factor (3).

### 2. Magnetic Cell Sorting and Flow Cytometry

CTCs in patient blood samples were separated with magnetic beads conjugated with anti-EpCAM antibodies (Miltenyi Biotec, Cologne, Germany) followed by positive magnetic selection according to the manufacturer's instructions. The resulting CTC-enriched samples were evaluated for CTC count and CTC phenotypes with a FACS Aria II machine (BD Biosciences, Franklin Lakes, N.J.), after immunostaining with EpCAM-APC antibody (Miltenyi Biotec, Cologne, Germany), CD44-PE Cy7 antibody, CD24-FITC antibody, and CD45-Horizon-V500 antibody (BD Biosciences, Franklin Lakes, N.J.). MCF-7 (EpCAM⁺CD24⁺) cells and MDA-MB-231 (EpCAM⁺CD44⁺) cells were spiked into human whole blood as controls. In some experiments, CTCs were sorted and their phenotypes were confirmed with confocal or fluorescence microscopy.

### 3. Negative Selection of Leukocytes

PBMC from patient blood was prepared as described above. If necessary red blood cells were lysed with ACK lysing buffer (Life Technologies, Carlsbad, Calif.) following the manufacturer's instructions. CD45 is a common leukocyte marker used to distinguish leukocytes. Thus, the cells were incubated with biotinylated human CD45 selection antibody (25 μl per 10⁷ cells) for 15 min at 4° C., followed by MagCellect™ (R&D Systems, Minneapolis, Minn.) Streptavidin Ferrofluid magnetic beads (50 μl per 10⁷ cells) for 15 minutes at 4° C. At the end of the incubation period, the cell suspension was washed with 9 ml of cold PlusCellect™ (R&D Systems, Minneapolis, Minn.) Buffer and centrifuged at 300×g for 8 minutes. The supernatant was pipetted out and discarded (eliminating a large fraction of unbound beads), and the cells in the pellet were retained, resuspended in 1 ml of cold PlusCellect™ buffer and were placed in a microfuge tube and incubated for 6+ minutes on the MagCellect™ (R&D Systems, Minneapolis, Minn.) magnet at room temperature. Cells not pulled out by the magnet were retained for staining. The cells were centrifuged and stained for EpCAM, CD44, CD24, and/or CD45, as described above.

### 4. CellSearch Assay

Tumor cells in mouse whole blood or in CTC cultures were enumerated using the CellSearch® System (Veridex Corporation, Warrenton, N.J.) following the manufacture's instruction. Ki-67 expression in CTCs was measured as a marker of cell proliferation (BD Biosciences, Franklin Lakes, N.J.).

### 5. DepArray Assay CTC

Cells in CTC cultures and single cell suspensions prepared from CTC-derived tumor xenografts were immunostained with EpCAM-PE antibody (Miltenyi Biotec, Cologne, Germany or R&D Biosystems, Minneapolis, Minn.), CD44-APC antibody, CD24-FTIC antibody (BD Biosciences, Franklin Lakes, N.J.), CD45-PerCP Cy5.5 antibody (eBiosciences, San Diego, Calif.). Cell images were captured and analyzed using the DepArray™ (Silicon Biosystems, Bologna, Italy) instrument.

### 6. In Vivo Tumor Formation

Cells from MBC patient CTC cultures were suspended in phosphate buffered saline (PBS) mixed with high concentration matrigel (BD Biosciences, Franklin Lakes, N.J.) at 10 mg/ml. Each aliquot of 0.2 mL containing 650 cells was injected into the third mammary fat pad of 6-8 week-old NOD/SCID (Cg-Prkdc^{scid} I12rg^{tm1Wj1}/SzJ) mouse (The Jackson Laboratory, Bar Harbor, Me.). In the tumor serial transplantation study, 2×2 mm pieces of tumor tissue from CTC-derived tumor xenografts were implanted in the mammary fad pad of Beige Nude XID mice (n=6).

### 7. Tumor Xenografts and Mouse Organs Fixation and Histopathology

Mice were sacrificed and xenograft tumor, lung, liver, and kidney were removed and fixed in buffered 10% formalin for 24 h, and paraffin embedded (FFPE). Sample sections were stained with hematoxylin and eosin (H&E) for histopathological analysis following standard histopathological techniques.

### 8. Immunohistochemistry

FFPE tissue sections were treated with heat-induced epitope retrieval technique using citrate buffer, pH 6 and then incubated with anti-human mammaglobin antibody (Spring Bioscience, Pleasonton, Calif.) and rabbit anti-human MHC-1 marker antibody (Novus, Littleton, Colo.). Human breast tumor was used as positive controls for human mammaglobin and MHC marker. Immunodetection was conducted using the rabbit labelled HRP polymer secondary antibody (Dako EnVision, Carpinteria, Calif.) followed by diaminobenzidine. All samples were counterstained with hematoxylin.

### 9. Quantitative and Phenotypical Analysis of CTCs and Tumor Xenografts

Tumor cells in the mouse blood or in the CTC cultures were quantified by the CellSearch® System (Veridex Corporation, Warrenton, N.J.) as described previously (Riethdorf et al. 2007). Ki-67 expression in CTCs was detected using anti-Ki-67 FITC antibody (BD Biosciences, Franklin Lakes, N.J.). Expression of CD44 and CD24 in tumor cells in the CTC culture was assessed by the DepArray™ (Silicon Biosystems, Bologna, Italy) instrument using anti-EpCAM PE antibody (Miltenyi Biotec, Cologne, Germany or R&D Biosystems, Minneapolis, Minn.), anti-CD44 APC antibody, anti-CD24 FTIC antibody (BD Biosciences, Franklin Lakes, N.J.), and anti-CD45 PerCP Cy5.5 antibody (eBiosciences, San Diego, Calif.). Single cell suspensions were prepared from the CTC-derived tumor xenografts by enzymatic dissociation with collagenase type 3, filtered (Worthington Biochemical Corp.). Phenotypic analysis of the tumor cells were conducted with the LSR II Flow Cytometer machine (BD Biosciences, Franklin Lakes, N.J.) and the DepArray™ (Silicon Biosystems, Bologna, Italy) instrument using anti-EpCAM PerCp Cy5.5 antibody (BD Biosciences, Franklin Lakes, N.J.), anti-CD44 APC antibody (BD Pharmingen, San Jose, Calif.), anti-CD24-FITC antibody (BD Pharmingen, San Jose, Calif.), and anti-H-2Kd PE antibody (BD Biosciences, Franklin Lakes, N.J.).

### 10. Quantitative RT-PCR Analysis of Xenograft and Mouse Organs

RNA was extracted from tumor cells in the CTC cultures, CTC-derived tumor xenografts, and mouse lung, liver and kidney samples using the RNeasy® (Qiagen, Germantown, Md.) Mini Kit, following the manufacturer's protocol. qPCR was performed on the 96.96 Dynamic Array™ (Fluidigm, South San Francisco, Calif.), as described previously (Huang et al. 2011).

### Example 1

### Evaluation of CTC Phenotypes

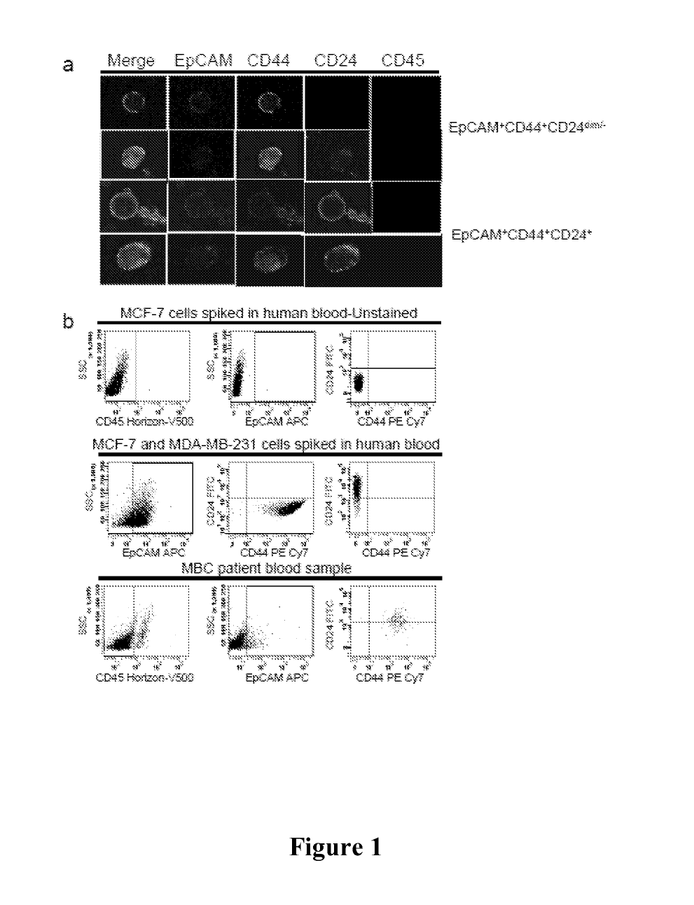

To determine whether breast cancer CTCs contain a CSC-like subpopulation, the phenotypes of CTCs from metastatic breast cancer (MBC) patients were assessed for expression of CD44 and CD24. Peripheral blood samples collected from MBC patients were enriched for CTCs using magnetic beads conjugated with an EpCAM-specific antibody and the resulting cell population was sorted by fluorescent-activated cell sorting (FACS) following immunostaining for EpCAM, CD44, CD24, and CD45. CD45 is a common leukocyte marker used to distinguish leukocytes. The phenotypes of the sorted CTCs were verified by confocal or fluorescence microscopy. CTCs from MBC patients showed heterogeneous phenotypes, including the CSC phenotype (EpCAM⁺CD44⁺CD24^{dim/-}) as well as other phenotypes such as EpCAM⁺CD44⁻CD24⁻CD45⁻ and EpCAM⁺CD44⁺CD24⁺CD45⁻ (FIGS. 1a and 1b). The prevalence of the CSC-like CTCs (EpCAM⁺CD44⁺CD24^{dim/-} cells) in total CTCs (EpCAM⁺CD45⁻) was assessed in five MBC patient samples that each contained greater than 100 CTCs. This CSC-like EpCAM⁺CD44⁺CD24^{dims/-} subpopulation was found in all patient samples analyzed and ranged from 4.6% to 71% of the total CTC population (FIG. 1b), as summarized in Table 1 below.

TABLE 1
Prevalence of CSC-like CTCs in breast cancer patients

| MBC | CTC number | CSC number | % of |
|---|---|---|---|
| Patient | (EpCAM⁺CD45⁻) | (EpCAM⁺CD44⁺CD24⁻CD45⁻) | CTC |
| B81 | 120 | 42 | 35.0 |
| 194 | 553 | 224 | 40.5 |
| 167 | 109 | 5 | 4.6 |
| 2F1 | 105 | 52 | 49.5 |
| 174 | 451 | 322 | 71.4 |
| Mean | 267 | 129 | 40.2 |

Disseminated tumor cells are frequently detected in the bone marrow of cancer patients with metastasis (Lacroix M, 2006). The percentage of CD44⁺CD24^{dim/-} CSC cells in bone marrow metastases of MBC patients was shown to be 71% (Balic et al., 2006). Studies by Abraham et al have demonstrated that CD44⁺CD24^{dim/-} CSC cells in the primary breast tumors are less than 10% of total tumor cells (Abraham et al., 2005). The prevalence of CSC-like CTCs observed in this study was consistent with that reported for primary breast tumors in the previous studies.

### Example 2

### In Vitro Culture of CTCs

To further characterize CTCs from breast cancer patients, attempts were made to grow CTCs in vitro. In initial experiments, CTCs in patient blood samples were sorted by FACS using the markers EpCAM, CD44, CD24, and CD45 after enrichment with anti-EpCAM antibody-coated magnetic beads. However, these sorted CTCs, including CSC-like CTCs, did not survive when placed in culture. CTCs are noted as being generally fragile and many of them undergo apoptosis and have a short half life (Meng et al., 2004; Mehes et al., 2001).

In order to maintain CTC viability and reduce CTC loss due to cell separation, PBMCs prepared from MBC patient blood samples, which were pre-selected to have greater than 100 CTCs/7.5 ml blood via the CellSearch® (Veridex Corporation, Warrenton, N.J.) method, were placed directly in culture without FACS sorting. The PBMCs were cultured using conditions reported to support and enrich for CSCs obtained from solid tumors following FACS sorting Briefly, cells were suspended in MammoCult® (Stem Cell Technologies, Vancouver, Canada) medium supplemented with MammoCult® (Stem Cell Technologies, Vancouver, Canada) proliferation supplements. The cells were subsequently cultured in 1 ml at a density of ˜2e6 cells/well in 6-well ultra-low attachment plates (Corning Inc., Corning, N.Y.) in a 5% CO₂ humidified incubator at 37° C. Every alternate day ˜500 μl media was added to each well in a 6-well plate until the cells were harvested for future experiments. The serum-free CSC medium preserves CSCs and may inhibit other cell growth. The number of leukocytes in this culture decreased over time. If the patient blood samples contained a large number of leukocytes, CD45 antibodies and magnetic beads were used in a negative selection step to remove leukocytes from the sample before suspending the cells in the cell culture medium.

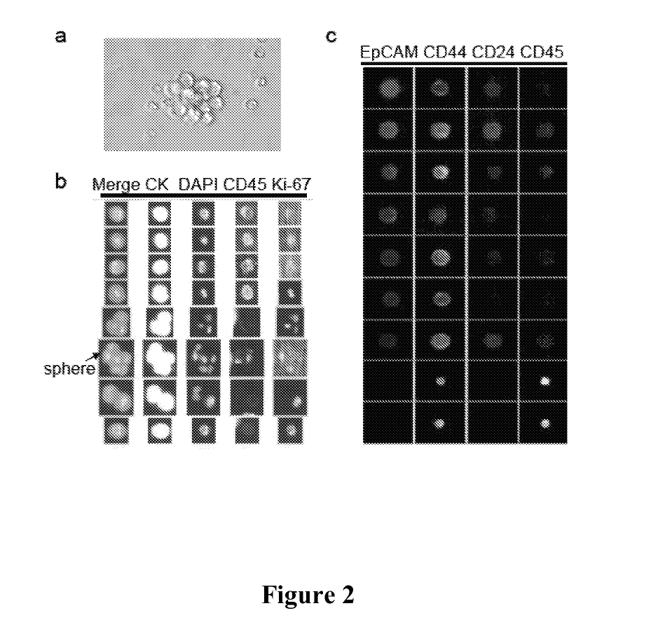

Tumor cells in the in vitro cultures were measured by the CellSearch® (Veridex Corporation, Warrenton, N.J.) method as well as immunostaining for EpCAM and CD45. In vitro culturing of CTCs was repeated from different patients after various times in culture; the longest duration in culture was 28 days (FIG. 2b). Phenotypic analysis demonstrated that the majority of CTCs in culture had the CSC (EpCAM⁺CD44⁺CD24^{dim/-}CD45⁻) surface marker phenotype despite the heterogeneity of the original sample (FIG. 2c). This suggests that these culture conditions preferentially favored CSC-like CTC outgrowth. Although the total number of tumor cells in the cultures did not significantly increase, some tumor cells in culture expressed the proliferation marker Ki-67, indicating that they were propagating (FIG. 2b). In some cases, the tumor cells in culture formed small multicellular spheres, a known property of CSCs (FIGS. 2a and 2b) (Ponti et al. 2006).

### Example 3

### In Vivo Tumor Formation

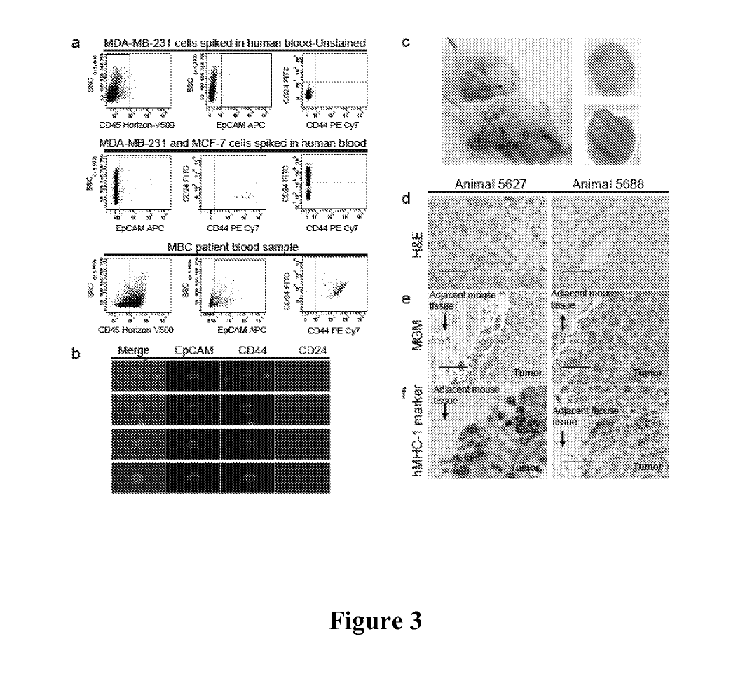

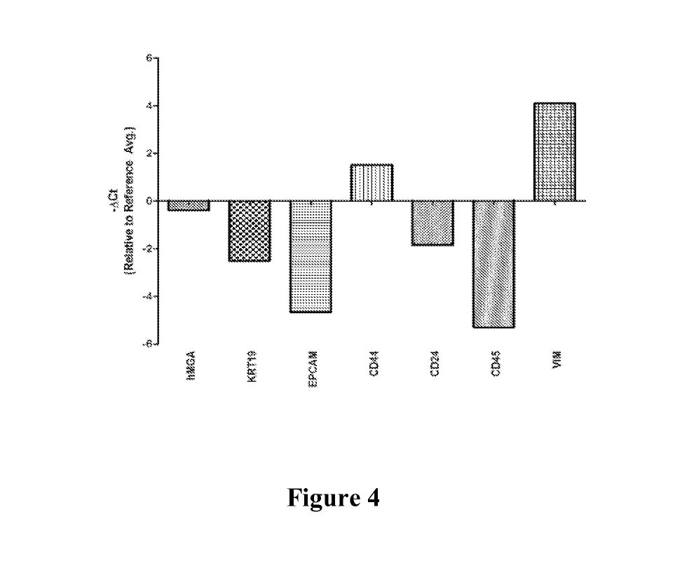

CSC tumorigenicity studies are generally conducted by injecting between 100 and 1000 cells into an immunocompromised mouse (Farrar W L, 2010). The majority of MBC patients have less than 100 CTCs per 7.5 ml blood, and only a subset of these are CSC-like CTCs (Allard et al., 2004). The low frequency of CSC-like CTCs in patient blood samples makes it challenging to assess in vivo tumorigenicity of CSC-like CTCs and has not been reported. To overcome this challenge, a peripheral blood sample from a MBC patient was selected that contained 446 CTCs (EpCAM⁺CD45⁻) in 7.5 ml of blood, of which 322 CTCs (71%) were EpCAM⁺CD44⁺CD24^{dim/-}CD45⁻ by FACS analysis (FIG. 3a). This sample was cultured for 9 days under CSC conditions (See Example 2), after which the majority of leukocytes died. Cells from this culture were analyzed 5 and 9 days after culture initiation, and all tumor cells were found to express the CSC phenotype of EpCAM⁺CD44⁺CD24^{dim/-} CD45⁻ by immunostaining (FIG. 3b). Additionally, gene expression analysis of tumor cells from the 9-day culture showed expression of high levels of CD44, human mammaglobin A (hMGA) and vimentin mRNAs, and a low level of CD24 mRNA, verifying that they were breast cancer tumor cells with a CSC phenotype (FIG. 4).

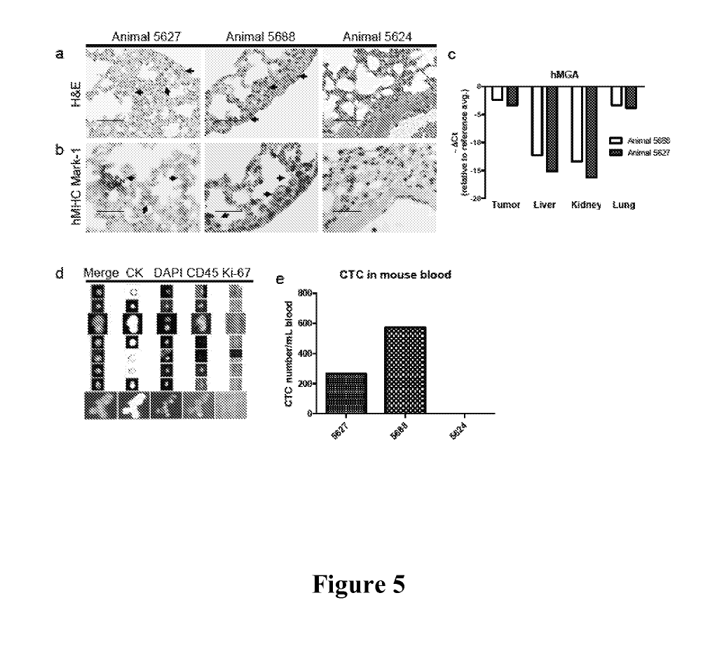

About 650 cells from the 9-day culture were injected into the mouse mammary fat pad of an immunodeficient mouse to assess tumorigenic potential of the cultured, CSC-like CTCs. Tumors developed at the injection sites in 2 of the 3 mice who received the cultured CSC-like CTCs (FIG. 3c). These CTC-derived tumor xenografts were removed from the mice for histopathological analysis ten months after cell implantation. The morphology of the tumors, as assessed by hematoxylin and eosin (H&E) staining, was consistent with a breast cancer origin (FIG. 3d). To further verify the human breast cancer origin of the tumors, the expression of human mammaglobin genes in the CTC-derived tumor xenografts was examined Immunohistochemical (IHC) analysis demonstrated expression of human mammaglobin protein in tumor cells of the xenografts but not in the adjacent mouse tissues (FIG. 3e). Human mammaglobin A (MGA) mRNA was also detected in the tumor xenografts (FIG. 5c).

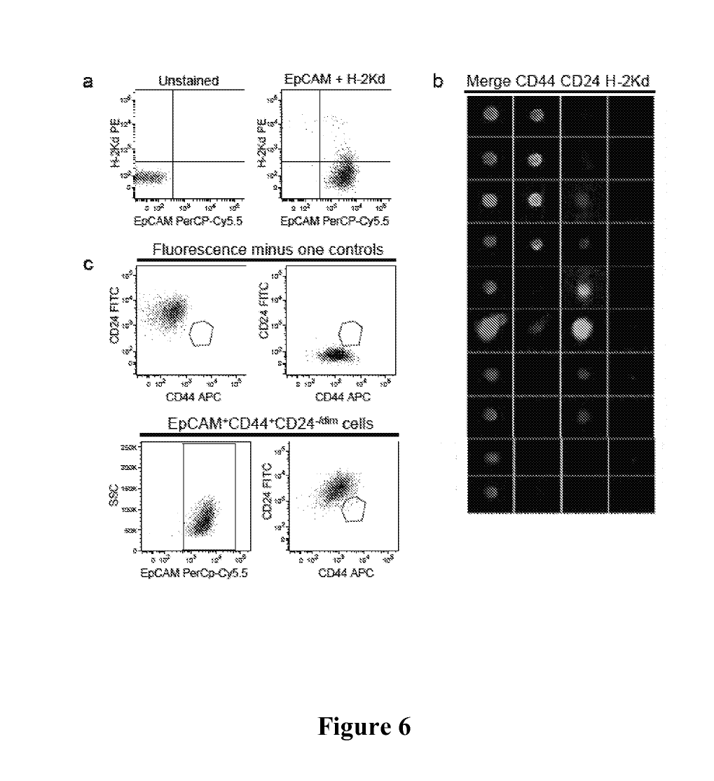

To exclude the possibility of spontaneous mouse tumor growth, expression of human and mouse Major Histocompatibility Complex (MHC) markers within the tumor xenografts were assessed. Human MHC-1 marker was expressed in tumor cells but not in the adjacent mouse tissues, as determined by IHC (FIG. 3f). In contrast, mouse MHC class I H-2Kd was not detected in the EpCAM-positive tumor xenografts by flow cytometric analysis (FIG. 6a).

The cellular phenotypes of the CTC-derived tumor xenografts were studied by dissociating tumor tissues and immunostaining for EpCAM, CD44, and CD24. The phenotypes of the tumor xenografts were heterogeneous (FIG. 6b). EpCAM⁺CD44⁺CD24^{dim/-} CSC-like cells accounted for only 0.8% and 2.7% of total tumor cells (FIG. 6c). Because the in vitro cultured CTCs that were implanted into the mice had a single phenotype of EpCAM⁺CD44⁺CD24^{dim/-}, the heterogeneous cell populations of the tumor xenografts suggest that the EpCAM⁺CD44⁺CD24^{dim/-} cells not only self renewed but also gave rise to other subpopulations of tumor cells during tumor formation and growth.

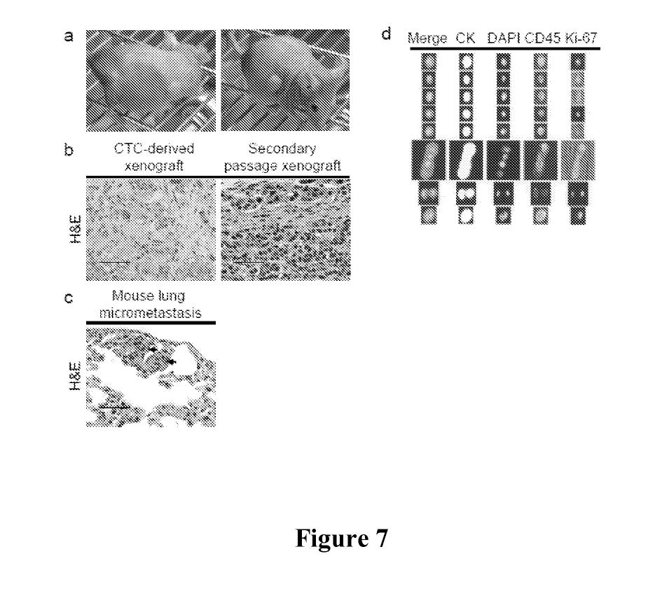

To determine whether the CTC-derived tumors could initiate new tumors, a hallmark of CSCs, small pieces of tumor xenograft tissue from both of the tumor-bearing mice were reimplanted into naïve, immunodeficient mice (n=6). Three months after the implantation, all of the implanted animals had grown new tumors, except for one animal that died early (FIG. 7). The secondary-passage tumor xenografts resembled histopathological features of the parent CTC-derived tumors (FIG. 7b). These findings indicate that the MBC CSC-like CTCs have the CSC characteristics of self-renewal, asymmetric cell division, and tumorigenicity, as do CSCs present within primary tumors.

### Example 4

### Cancer Metastasis In Vivo

To determine whether CTC-derived tumor xenografts were able to metastasize, lung, liver, and kidney were collected and examined for tumor lesions from all three mice that received the initial implantation of the cultured CSC-like CTCs. Micrometastases were found in lungs from the two mice that had primary tumors, but not in the lungs of the tumor-free mouse (FIG. 5a). The metastatic tumor lesions in the lung were shown to be of human breast cancer origin by immunostaining for the human MHC-1 marker (FIG. 5b) and RT-PCR analysis of expression of human MGA mRNA (FIG. 5c). No metastasis was observed in the livers or kidneys from any of the three mice.

CTCs and CTC clusters were detected in the peripheral blood of the two mice that grew tumors (267 CTCs/mL whole blood in one mouse and 573 CTCs/mL whole blood in the other), but not in the mouse without tumor growth (FIG. 5d). Detection of CTC clusters in cancer patient blood has been associated with increased metastasis (Yu et al., 2011). Furthermore, lung metastatic tumor lesions were also observed in the mice that were implanted with the CTC-derived tumor xenograft tissue (FIG. 7c); CTCs and CTC clusters were detected in the blood of these mice (FIG. 7d).

These results indicate that a CSC-like population within CTCs derived from MBC patients was able to initiate tumor formation and metastasize to the lungs in mice. Furthermore, the tumors formed retained CSC-like cells and disseminated tumor cells into circulation in mice. Additional studies will help determine whether other subsets of CTCs have the similar tumorigenic and metastatic activities.

Metastasis accounts for 90% of cancer deaths (Weigelt, B. et al. 2005). These results suggest that the blockade of metastasis by targeting a CSC-like population within CTCs has the potential to improve cancer therapy and furthermore, provides a minimally invasive method to evaluate this population in human cancer patients. The CSC-like population within CTCs also provides a readily accessible biomarker for tumors and the evaluation of therapies in cancer patients.

### EMBODIMENTS

1. A method of culturing cancer stem cells in vitro, the method comprising:
   a. preparing a cell culture by incubating in vitro peripheral blood mononuclear cells (PBMCs) in a serum-free cell culture medium suitable for supporting cancer stem cell maintenance, wherein the PBMCs are obtained from a carcinoma patient and comprise cancer stem cells;
   b. maintaining the cell culture in the serum-free cell culture medium for at least 5-28 days to obtain an enriched population of cancer stem cells.
2. The method of embodiment 1 wherein the cell culture is maintained in the serum-free cell culture medium for 5-9 days.
3. The method of embodiment 2 wherein the cell culture is maintained in the serum-free cell culture medium for 9 days.
4. The method of embodiment 1, wherein after culturing in vitro for at least 5-28 days, the population of cancer stem cells in the cell culture relative to the PBMCs is enriched at least 10,000-fold.
5. The method of any one of embodiments 1-4, wherein flow cytometry is not used to obtain the enriched population of cancer stem cells.
6. The method of any of embodiments 1-5 wherein the serum-free medium is a cancer stem cell media.
7. The method of embodiment 6 wherein the cancer stem cell media is mTeSR1.
8. The method of any of embodiments 1-7 wherein prior to incubating the PBMCs in the serum-free cell culture medium, the PBMCs are treated to remove leukocytes.
9. The method of any of embodiments 1-6 and 8, wherein the carcinoma patient is a breast cancer patient.
10. The method of embodiment 9 wherein the serum-free media is Mammocult media.
11. An enriched population of cancer stem cells obtained according to the embodiment of any one of claims 1-10.
12. A method of forming a human tumor in an immunodeficient non-human mammal, the method comprising injecting an enriched population of human cancer stem cells from a carcinoma patient into the immunodeficient non-human mammal, wherein the enriched population of human cancer stem cells were obtained from the peripheral blood of the carcinoma patient and wherein the injected cells form the human tumor in the immunodeficient non-human mammal
13. The method of embodiment 12, further comprising before the injection step:
   a. preparing a cell culture by incubating in vitro peripheral blood mononuclear cells (PBMCs) obtained from the carcinoma patient in a serum-free cell culture medium suitable for supporting cancer stem cells in culture, wherein the PBMCs comprise human cancer stem cells;
   b. maintaining the cell culture in the serum-free cell culture medium for at least 5-28 days to obtain the enriched population of human cancer stem cells.
14. The method of embodiment 13 wherein the cell culture is maintained in the serum-free cell culture media for 5-9 days.
15. The method of embodiment 14 wherein the cell culture is maintained in the serum-free cell culture media for 9 days.
16. The method of any embodiments 13-15 wherein the population of human cancer stem cells injected in the mammal relative to that in the PBMCs obtained from the carcinoma patient is enriched for cancer stem cells at least 10,000-fold.
17. The method of any of embodiments 13-16, wherein flow cytometry is not used to obtain the enriched population of cancer stem cells.
18. The method of any of embodiments 13-17 wherein the cell culture media is a cancer stem cell media.
19. The method of embodiment 18 wherein the cancer stem cell media is mTeSR1.
20. The method of any of embodiments 12-19 wherein the carcinoma patient is a breast cancer patient.
21. The method of any one of embodiments 12-20, further comprising a step of isolating the human tumor formed in the immunodeficient non-human mammal and injecting cancer cells obtained from the isolated human tumor into a second immunodeficient non-human mammal, wherein the injected cancer cells obtained from the isolated human tumor form a second human tumor in the second immunodeficient non-human mammal
22. A method for determining the effectiveness of a test compound on reducing the number or activity of cancer stem cells from a carcinoma patient, the method comprising:
   a. injecting an enriched population of cancer stem cells from the carcinoma patient into an immunodeficient non-human mammal, wherein the cancer stem cells were obtained from the peripheral blood of the carcinoma patient;
   b. administering the test compound to the immunodeficient non-human mammal before, after, or at the same time as the cancer stem cells are injected into the immunodeficient non-human mammal;
   c. determining the number or activity of cancer stem cells in the immunodeficient non-human mammal; and
   d. comparing the activity or number of cancer stem cells in the immunodeficient non-human mammal to a control non-human mammal, wherein a reduction in the activity or number of cancer stem cells in the immunodeficient non-human mammal as compared to the control non-human mammal indicates that the test compound is effective to reduce the activity or number of the cancer stem cells from the carcinoma patient.
23. The method of embodiment 22 wherein the effectiveness of the test compound is determined by activity of the cancer stem cells, and wherein the activity is tumor formation.
24. The method of embodiment 22 wherein the effectiveness of the test compound is determined by activity of the cancer stem cells, and wherein the activity is metastasis.
25. The method of embodiment 22 wherein the effectiveness of the test compound is determined by reducing the number of cancer stem cells.
26. The method of any of embodiments 22-25, further comprising before the injection step:
   a. preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells;
   b. maintaining the cell culture in the serum-free cell culture medium for at least 5-28 days to obtain the enriched population of cancer stem cells.
27. The method of embodiment 26 wherein the cell culture is maintained in the serum-free cell culture media for 5-9 days.
28. The method of embodiment 27 wherein the cell culture is maintained in the serum-free cell culture media for 9 days.
29. The method of any of embodiments 26-28, wherein flow cytometry is not used to obtain the enriched population of cancer stem cells.
30. The method of any of embodiments 26-29 wherein prior to incubating the PBMCs in the serum-free cell culture media the PBMCs are treated to remove leukocytes.
31. The method of any of embodiments 26-30 wherein the serum-free cell culture medium is a cancer stem cell media.
32. The method of embodiment 31 wherein the cancer stem cell media is Mammocult or mTeSR.
33. An in vitro method for measuring the effect of a test compound on cancer stem cells from a carcinoma patient, the method comprising:
   a. adding the test compound to an in vitro culture of cancer stem cells, wherein the cancer stem cells were obtained from the peripheral blood of the carcinoma patient;
   b. measuring the effect of the test compound on the cancer stem cells.
34. The method of embodiment 33, further comprising before the adding step:
   a. preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells;
   b. maintaining the cell culture in the serum-free cell culture medium for at least 5-28 days to obtain an enriched population of cancer stem cells.
35. The method of embodiment 34 wherein cell culture is maintained in the serum-free cell culture medium for 5-9 days.
36. The method of embodiment 35 wherein the cell culture is maintained in the serum-free cell culture medium for 9 days.
37. The method of any of embodiments 34-36 wherein the serum-free culture medium is a cancer stem cell medium.
38. The method of claim of embodiments 34-37 wherein prior to incubating the PBMCs in the serum-free medium the PBMCs are treated to remove leukocytes.
39. The method of any of embodiments 34-38, wherein flow cytometry is not used to obtain the enriched population of cancer stem cells.
40. The method of any of embodiments 34-39 wherein after culturing in vitro for at least 5-28 days, the population of cancer stem cells in the cell culture relative to the PBMCs is enriched at least 10,000-fold.
41. The method of any of embodiments 33-40 wherein the effect of the test compound on the cancer stem cells is a decrease in the number of cancer stems relative to a control population of cancer stem cells not treated with the test compound
42. The method of any of embodiments 33-41 wherein the carcinoma patient is a breast cancer patient.

The following references are cited in the application and provide general information on the field of the invention and provide assays and other details discussed in the application. The following references are incorporated herein by reference in their entirety.
Fidler, I. J. The pathogenesis of cancer metastasis: the ‘seed and soil’ hypothesis revisited. Nat. Rev. Cancer 3, 453-458 (2003).
Lawson, J. C., Blatch, G. L. & Edkins, A. L. Cancer stem cells in breast cancer and metastasis. Breast Cancer Res. Treat. 118, 241-254 (2009).
Kasimir-Bauer, S., Hoffmann, O., Wallwiener, D., Kimmig, R. & Fehm, T. Expression of stem cell and epithelial-mesenchymal transition markers in primary breast cancer patients with circulating tumor cells. Breast Cancer Res. 14, R15 (2012).
Aktas, B. et al. Stem cell and epithelial-mesenchymal transition markers are frequently overexpressed in circulating tumor cells of metastatic breast cancer patients. Breast Cancer Res. 11, R46 (2009).
Mego, M., Mani, S. A. & Cristofanilli, M. Molecular mechanisms of metastasis in breast cancer—clinical applications. Nat. Rev. Clin. Oncol. 7, 693-701 (2010).
Kang, Y. et al. A multigenic program mediating breast cancer metastasis to bone. Cancer Cell 3, 537-549 (2003).
Liu, R. et al. The prognostic role of a gene signature from tumorigenic breast-cancer cells. N Engl. J. Med. 356, 217-226 (2007).
Landemaine, T. et al. A six-gene signature predicting breast cancer lung metastasis. Cancer Res. 68, 6092-6099 (2008).
Al-Hajj, M., Wicha, M. S., ito-Hernandez, A., Morrison, S. J. & Clarke, M. F. Prospective identification of tumorigenic breast cancer cells. Proc. Natl. Acad. Sci. U.S.A 100, 3983-3988 (2003).
Abraham, B. K. et al. Prevalence of CD44+/CD24−/low cells in breast cancer may not be associated with clinical outcome but may favor distant metastasis. Clin. Cancer Res. 11, 1154-1159 (2005).
Sheridan, C. et al. CD44+/. Breast Cancer Res. 8, R59 (2006).
Balic, M. et al. Most early disseminated cancer cells detected in bone marrow of breast cancer patients have a putative breast cancer stem cell phenotype. Clin. Cancer Res. 12, 5615-5621 (2006).
Liu, H. et al. Cancer stem cells from human breast tumors are involved in spontaneous metastases in orthotopic mouse models. Proc. Natl. Acad. Sci. U.S.A 107, 18115-18120 (2010).
Lacroix, M. Significance, detection and markers of disseminated breast cancer cells. Endocr. Relat Cancer 13, 1033-1067 (2006).
Meng, S. et al. Circulating tumor cells in patients with breast cancer dormancy. Clin. Cancer Res. 10, 8152-8162 (2004).
Mehes, G., Witt, A., Kubista, E. & Ambros, P. F. Circulating breast cancer cells are frequently apoptotic. Am. J. Pathol. 159, 17-20 (2001).
Ponti, D., Zaffaroni, N., Capelli, C. & Daidone, M. G. Breast cancer stem cells: an overview. Eur. J. Cancer 42, 1219-1224 (2006).
Farrar W. L. Cancer Stem Cells. Cambridge University Press, (2010).
Allard, W. J. et al. Tumor cells circulate in the peripheral blood of all major carcinomas but not in healthy subjects or patients with nonmalignant diseases. Clin. Cancer Res. 10, 6897-6904 (2004).
Yu, M. et al. Circulating tumor cells: approaches to isolation and characterization. J. Cell Biol. 192, 373-382 (2011).
Weigelt, B., Peterse, et al. Breast cancer metastasis: markers and models. Nat. Rev. Cancer 5, 591-602 (2005).
Huang, J. et al. Altered expression of insulin receptor isoforms in breast cancer. PLoS. One. 6, e26177 (2011).
Brehm et al. Humanized mouse models to study human diseases Curr Opin Endocrinol Diabetes Obes. 2, 120-125 (2010)

What is claimed:

1. A method of culturing cancer stem cells in vitro, the method comprising:
a. preparing a cell culture by incubating in vitro peripheral blood mononuclear cells (PBMCs) in a serum-free cell culture medium suitable for supporting cancer stem cell maintenance, wherein the PBMCs are obtained from a carcinoma patient and comprise cancer stem cells;
b. maintaining the cell culture in the serum-free cell culture medium for at least 5-28 days to obtain an enriched population of cancer stem cells.

2. The method of claim 1 wherein the cell culture is maintained in the serum-free cell culture medium for 5-9 days.

3. The method of claim 2 wherein the cell culture is maintained in the serum-free cell culture medium for 9 days.

4. The method of claim 1, wherein after culturing in vitro for at least 5-28 days, the population of cancer stem cells in the cell culture relative to the PBMCs is enriched at least 10,000-fold.

5. The method of any one of claims 1-4, wherein flow cytometry is not used to obtain the enriched population of cancer stem cells.

6. The method of any of claims 1-5 wherein the serum-free medium is a cancer stem cell media.

7. The method of claim 6 wherein the cancer stem cell media is mTeSR1.

8. The method of any of claims 1-7 wherein prior to incubating the PBMCs in the serum-free cell culture medium, the PBMCs are treated to remove leukocytes.

9. The method of any of claims 1-6 and 8, wherein the carcinoma patient is a breast cancer patient.

10. The method of claim 9 wherein the serum-free media is Mammocult media.

11. An enriched population of cancer stem cells obtained according to the claim of any one of claims 1-10.

12. A method of forming a human tumor in an immunodeficient non-human mammal, the method comprising injecting an enriched population of human cancer stem cells from a carcinoma patient into the immunodeficient non-human mammal, wherein the enriched population of human cancer stem cells were obtained from the peripheral blood of the carcinoma patient and wherein the injected cells form the human tumor in the immunodeficient non-human mammal.

13. The method of claim 12, further comprising before the injection step:
a. preparing a cell culture by incubating in vitro peripheral blood mononuclear cells (PBMCs) obtained from the carcinoma patient in a serum-free cell culture medium suitable for supporting cancer stem cells in culture, wherein the PBMCs comprise human cancer stem cells;
b. maintaining the cell culture in the serum-free cell culture medium for at least 5-28 days to obtain the enriched population of human cancer stem cells.

14. The method of claim 13 wherein the cell culture is maintained in the serum-free cell culture media for 5-9 days.

15. The method of claim 14 wherein the cell culture is maintained in the serum-free cell culture media for 9 days.

16. The method of any claims 13-15 wherein the population of human cancer stem cells injected in the mammal relative to that in the PBMCs obtained from the carcinoma patient is enriched for cancer stem cells at least 10,000-fold.

17. The method of any of claims 13-16, wherein flow cytometry is not used to obtain the enriched population of cancer stem cells.

18. The method of any of claims 13-17 wherein the cell culture media is a cancer stem cell media.

19. The method of claim 18 wherein the cancer stem cell media is mTeSR1.

20. The method of any of claims 12-19 wherein the carcinoma patient is a breast cancer patient.

21. The method of any one of claims 12-20, further comprising a step of isolating the human tumor formed in the immunodeficient non-human mammal and injecting cancer cells obtained from the isolated human tumor into a second immunodeficient non-human mammal, wherein the injected cancer cells obtained from the isolated human tumor form a second human tumor in the second immunodeficient non-human mammal.

22. A method for determining the effectiveness of a test compound on reducing the number or activity of cancer stem cells from a carcinoma patient, the method comprising:
a. injecting an enriched population of cancer stem cells from the carcinoma patient into an immunodeficient non-human mammal, wherein the cancer stem cells were obtained from the peripheral blood of the carcinoma patient;
b. administering the test compound to the immunodeficient non-human mammal before, after, or at the same time as the cancer stem cells are injected into the immunodeficient non-human mammal;
c. determining the number or activity of cancer stem cells in the immunodeficient non-human mammal; and
d. comparing the activity or number of cancer stem cells in the immunodeficient non-human mammal to a control non-human mammal, wherein a reduction in the activity or number of cancer stem cells in the immunodeficient non-human mammal as compared to the control non-human mammal indicates that the test compound is effective to reduce the activity or number of the cancer stem cells from the carcinoma patient.

23. The method of claim 22 wherein the effectiveness of the test compound is determined by activity of the cancer stem cells, and wherein the activity is tumor formation.

24. The method of claim 22 wherein the effectiveness of the test compound is determined by activity of the cancer stem cells, and wherein the activity is metastasis.

25. The method of claim 22 wherein the effectiveness of the test compound is determined by reducing the number of cancer stem cells.

26. The method of any of claims 22-25, further comprising before the injection step:
a. preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells;
b. maintaining the cell culture in the serum-free cell culture medium for at least 5-28 days to obtain the enriched population of cancer stem cells.

27. The method of claim 26 wherein the cell culture is maintained in the serum-free cell culture media for 5-9 days.

28. The method of claim 27 wherein the cell culture is maintained in the serum-free cell culture media for 9 days.

29. The method of any of claims 26-28, wherein flow cytometry is not used to obtain the enriched population of cancer stem cells.

30. The method of any of claims 26-29 wherein prior to incubating the PBMCs in the serum-free cell culture media the PBMCs are treated to remove leukocytes.

31. The method of any of claims 26-30 wherein the serum-free cell culture medium is a cancer stem cell media.

32. The method of claim 31 wherein the cancer stem cell media is Mammocult or mTeSR.

33. An in vitro method for measuring the effect of a test compound on cancer stem cells from a carcinoma patient, the method comprising:
a. adding the test compound to an in vitro culture of cancer stem cells, wherein the cancer stem cells were obtained from the peripheral blood of the carcinoma patient;
b. measuring the effect of the test compound on the cancer stem cells.

34. The method of claim 33, further comprising before the adding step:
a. preparing a cell culture by incubating in vitro peripheral blood mononuclear cells (PBMCs) obtained from the carcinoma patient in a serum-free cell culture medium suitable for supporting cancer stem cell maintenance, wherein the PBMCs comprise cancer stem cells;
b. maintaining the cell culture in the serum-free cell culture medium for at least 5-28 days to obtain an enriched population of cancer stem cells.

35. The method of claim 34 wherein cell culture is maintained in the serum-free cell culture medium for 5-9 days.

36. The method of claim 35 wherein the cell culture is maintained in the serum-free cell culture medium for 9 days.

37. The method of any of claims 34-36 wherein the serum-free culture medium is a cancer stem cell medium.

38. The method of claim of claims 34-37 wherein prior to incubating the PBMCs in the serum-free medium the PBMCs are treated to remove leukocytes.

39. The method of any of claims 34-38, wherein flow cytometry is not used to obtain the enriched population of cancer stem cells.

40. The method of any of claims 34-39 wherein after culturing in vitro for at least 5-28 days, the population of cancer stem cells in the cell culture relative to the PBMCs is enriched at least 10,000-fold.

41. The method of any of claims 33-40 wherein the effect of the test compound on the cancer stem cells is a decrease in the number of cancer stems relative to a control population of cancer stem cells not treated with the test compound

42. The method of any of claims 33-41 wherein the carcinoma patient is a breast cancer patient.
